(19) United States
(12) Patent Application Publication
(10) Pub. No.: US 2020/0338162 A1
(43) Pub. Date: Oct. 29, 2020

(54) EFFECT OF HEMOPEXIN THERAPY AFTER INTRACEREBRAL HEMORRHAGE

(71) Applicant: Thomas Jefferson University, Philadelphia, PA (US)

(72) Inventor: Raymond F. Regan, Villanova, PA (US)

(73) Assignee: Thomas Jefferson University, Philadelphia, PA (US)

(21) Appl. No.: 16/922,961

(22) Filed: Jul. 7, 2020

Related U.S. Application Data
(63) Continuation of application No. 15/735,732, filed on Dec. 12, 2017, now abandoned.
(60) Provisional application No. 62/175,027, filed on Jun. 12, 2015.

(51) Int. Cl. A61K 38/17; G01N 33/68; A61P 7/10; A61P 39/06; A61P 25/00; A61P 29/00

(52) U.S. Cl. CPC A61K 38/1709; G01N 33/6896; A61K 38/17; A61P 7/10; A61P 39/06; A61P 25/00; G01N 2800/7095; G01N 2800/28; G01N 2800/52; G01N 2333/4728; G01N 2333/47; G01N 2800/7009; A61P 29/00

(57) ABSTRACT

A method for providing perihematomal protection to a patient after suffering an TCH comprising administering an effective amount of Hx to said patient to increase serum concentrations to between two and three times normal serum levels, wherein said increased serum concentrations are maintained for between 3 and 21 days.

### PRIORITY CLAIM

This application claims the benefit of U.S. Provisional Application Ser. No. 62/175,027, filed Jun. 12, 2015. the disclosure content of which is hereby incorporated by reference in its entirety.

### GOVERNMENT SUPPORT

This invention was made with government support under Grant No. 1R21NS088986 and Grant No. NS079500 awarded by the National Institutes of Health. The government has certain rights in the invention.

### FIELD OF INVENTION

The present application is generally related to the serum protein hemopexin, with regard to the ability of hemopexin to be administered after intracerebral hemorrhage and protect cells around the hematoma to reduce breakdown of the blood-brain barrier.

### BACKGROUND OF THE INVENTION

Intracerebral hemorrhage (ICH) accounts for 10-15% of strokes, and is associated with significant mortality, morbidity, and economic cost. Therapy is currently limited to hematoma evacuation when indicated, reversal of anticoagulation, antihypertensive therapy, and supportive care. The inadequacy of this approach is demonstrated by mortality statistics, which are unchanged over the past two decades.

A growing body of experimental evidence suggests that toxins produced by the hematoma may contribute in delayed fashion to this poor outcome. One possible toxin is heme, which rapidly oxidizes to hemin when released into the extracellular space. The primary defense against extracellular heme or hemin is provided by hemopexin (Hx), a glycoprotein that binds them with extraordinary affinity and mitigates their pro-oxidant effect.

There are Few publications on hemopexin and its uses in vivo. One such publication is U.S. Patent Pub. No 2014/0249087, entitled “Use of Hemopcxin to Sequester Hemoglobin” which describes the use of hemopexin to treat or limit hemorrhage. Indeed, the '087 Pub. describes administering hemopexin to sequester at least 20% of extravascular hemoglobin to assist in reducing inflammation.

One small molecule study with deferoxamine is currently being tested for therapeutic significance. Although deferoxamine and Hx are not mutually exclusive therapeutics, and their preferred ligands differ, it is concerning that studies with DFO have not been successful. Indeed, a clinical trial of deferoxamine (DFO) after ICH is in progress (iDEF Trial, ClinicalTrials.gov Identifier NCT02175225) and is currently recruiting participants at 29 sites in the United States and Canada. A prior study using high-dose DFO (HI-DEF Trial, ClinicalTrials.gov Identifier NCT01662895) was halted before completion due to an increased incidence of acute respiratory distress syndrome (ARDS) in patients receiving DFO. ARDS after prolonged infusion of high-dose DFO was first observed over two decades ago [1]. The mechanism is undefined, but ARDS has not been reported to date as an adverse effect of other iron chelators in clinical use. No published study has linked elevated levels of serum hemopexin with ARDS. Conversely, protective effects of exogenous hemopexin in models of acute lung injury have been demonstrated [2,3]. The toxicity observed in the HI-DEF DFO trial does not have negative implications for the therapeutic use of hemopexin after ICH.

Applicant has discovered, and as described herein, that hemopexin contains additional, undescribed protective effects when particularly administered after intracerebral hemorrhage and provides for protection of the blood-brain barrier after intracerebral hemorrhage.

### SUMMARY OF THE INVENTION

In accordance with these and other objects, a first embodiment of an invention disclosed herein is directed to a method for administering hemopexin to a patient after suffering from an intracerebral hemorrhage, comprising administering an effective amount of hemopexin to a patient to protect cells around the hematoma and reduce the breakdown of the blood-brain barrier.

A further embodiment is related to a method for protecting perihematomal cells after ICH comprising administering an effective amount of hemopexin to a patient.

A further embodiment is related to a method for treating edema after ICH comprising administering an effective amount of hemopexin to a patient.

A further embodiment comprises a method for treating a patient after ICH injury comprising administering to a patient an effective dose of Hx to increase serum Hx levels in the patient to prevent perihematomal injury to the patient, including attenuation of injury to the blood-brain barrier.

A further embodiment comprises a method for treating a patient after ICH injury comprising administering to a patient an effective dose of Hx to increase serum Hx levels in the patient to prevent perihematomal injury to the patient, including attenuation of brain edema.

A further embodiment comprises a method for treating a patient after ICH injury comprising administering to a patient an effective dose of Hx to increase serum Hx levels in the patient to prevent perihematomal injury to the patient, including attenuation of brain cell injury adjacent to the hematoma.

A further embodiment comprises a method for treating a patient after ICH injury comprising administering to a patient an effective dose of Hx to increase serum Hx levels in the patient to prevent perihematomal injury to the patient, including attenuation of neurological deficits after ICH.

A further embodiment is directed to a method of administering hemopexin to a patient after ICH injury, comprising administering to said patient a sufficient dose of hemopexin to provide serum concentration between 1.2 and 3.6 mg/ml. In a preferred embodiment, methods of treatment are aimed to provide between about 2-3X normal serum Hx concentrations.

A further embodiment is directed to a method for treating a patient after ICH injury comprising administering to said patient a sufficient dose of hemopexin to provide for serum concentration between 1.2 to 3.6 mg/ml; providing two additional doses on consecutive days to maintain said serum concentration; and followed by treatment daily or on alternating days for five to ten additional doses.

A method for administration of Hemopexin (Hx) after Intracerebral hemorrhage (ICH) comprising administering to a patient an effective amount of Hx wherein said Hx is effective in reducing perihematomal cell injury, reducing edema, reducing inflammation and reducing neurological deficits after ICH.

A method for treating a patient after ICH comprising administering to said patient an effective amount of hemopexin between 1 to 72 hours after suffering from said ICH. In further embodiments, the Hx is administered to a patient for at least 10 days after the initial dose of hemopexin is administered. In further embodiments, the effective amount of Hx is sufficient to increase Hx serum levels to between about 2 and 3 times of the patient's normal serum Hx level.

In further methods, it is appropriate to first determine the patient's serum Hx levels so as to provide a baseline for determination of 2 and 3 times of the normal serum Hx level for said patient.

A method of treating a hematoma in the brain comprising: determining whether an ICH injury has occurred in the brain; determining the normal serum Hx levels of the patient; determining an increased serum concentration for the patient which is between two to three times the normal serum Hx level; determining an appropriate dose of Hx wherein the increased serum concentration for the patient will be reached within 48 hours. In certain embodiments, the increased serum concentration is achieved through two or more administrations of Hx given over the 48 hour period and wherein administration is completed through IV, or ventricular catheters or through intracerebroventricular infusion.

A method of reducing central nervous system injury in a patient after suffering an ICH by administering an effective amount of Hx to increase serum concentration to between 1.0 and 3.5 mg/ml, wherein said Hx downregulates the response of infiltrating inflammatory cells after the acute CNS injury.

A method of treating a patient with Hx after suffering an ICH comprising: determining an increased serum concentration for the patient which is between two to three times the normal serum Hx level; determining an appropriate dose of Hx wherein the increased serum concentration for the patient will be reached within 48 hours; and administering the Hx to the patient to so as to reach said increased serum Hx level. In certain embodiments, after the increased serum Hx level is reached in said patient, a maintenance phase is entered, wherein an effective amount of Hx is administered to said patient so as to maintain said increased serum Hx level for a duration sufficient for treatment of the hematoma.

A method of protecting cells around a hematoma after ICH to reduce breakdown of the blood-brain barrier, comprising: administering to a patient, an effective amount of Hx sufficient to raise the serum Hx levels in the patient to between two and three times normal physiological levels; measuring the serum Hx level at about 24 hours post administration; wherein the level of serum Hx is determined; and treating the patient with a further dose of Hx so as to achieve serum Hx levels of between two to three times normal physiological levels. In preferred embodiments the Hx levels between two and three times normal physiological levels are between 1.2 and 3.5 mg/ml.

A method of treatment of a patient after ICH comprising administering hemopexin to a patient so as to reduces hemin uptake by vulnerable cells and directs it to cell populations that robustly express LRP1 and are specialized for its catabolism (i.e. macrophages/microglia, hepatocytes); wherein the hemin-Hx complex induces the antioxidant/anti-inflammatory enzyme heme oxygenase-1; wherein the Hx directly inhibits neutrophil migration; and wherein the H also reduces macrophage TNF-α and IL-6 production in response to heme/hemin or lipopolysaccharide (LPS).

A method of treatment increasing perihematomal cell viability after ICH comprising: administering an effective amount of Hx to a patient within 24 hours of suffering from the ICH, wherein the effective amount of the Hx is sufficient to raise serum Hx concentrations to between about 1.2 and 3.5 mg/ml robust increase in perihematomal cell viability after ICH induction.

Use of hemopexin for treatment of intracerebral hemorrhage.

Use of hemopexin for reducing perihematomal cell injury, reducing edema, reducing inflammation, and reducing neurological deficits after ICH.

Use of hemopexin for treatment according to any one of the methods described herein.

### BRIEF DESCRIPTION OF THE DRAWINGS

FIG. 1 depicts mean striatal cell viability after intracerebral hemorrhage in mice treated with hemopexin (Hx) or PBS vehicle control via intraperitoneal (i.p.) or intranasal (i.n.) administration.

FIG. 2 depicts mean striatal cell viability after collagenase-induced ICH in hemopexin (Hx) knockout (KO) mice, wild-type (WT) mice, and wild-type mice treated with hemopexin at 2 hours after ICH.

FIG. 3 depicts hemopexin treatment after ICH attenuates blood-brain barrier injury, as measured by Evans blue leakage into the brain parenchyma.

FIG. 4 depicts that hemopexin blocks the neurotoxicity of oxidized heme (hemin) in vitro. Murine cortical cultures were treated with hemin 10 μM alone or with 1 mg/ml hemopexin (Hx). Cell injury was assessed by LDH release assay.

FIG. 5 depicts that Human hemopexin injection 70 mg/kg i.p. daily maintains serum concentration near target range of 0.6-1.2 mg/ml. This assay was specific for human hemopexin and does not measure native mouse hemopexin, which ranged from 0.5-1 mg/ml.

FIG. 6 depicts that lower dose hemopexin (Hx) therapy increases cell viability in the blood injection intracerebral hemorrhage model. Mice received Hx 35 mg/kg i.p. daily beginning 2 hours after striatal blood injection, and repeated daily. Striatal cell viability was quantified at 8 days with MTT assay. *P=0.014 v. mice treated with PBS vehicle.

FIG. 7 depicts that hemopexin therapy has no effect on striatal oxidized heme (hemin) content. Mice had intracerebral hemorrhage induced by collagenase injection, then were treated daily with 70 mg/kg Hx or PBS vehicle. Striatal hemin was assayed at days 3 and 7. These results indicate that hemopexin is not protecting by mobilizing and removing heme from the striatum.

### DETAILED DESCRIPTION OF THE PREFERRED EMBODIMENTS

The embodiments of the invention and the various features and advantages thereto are more fully explained with references to the non-limiting embodiments and examples that are described and set forth in the following descriptions of those examples. Descriptions of well-known components and techniques may be omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the appended claims.

As used herein, terms such as “a,” “an,” and “the” include singular and plural referents unless the context clearly demands otherwise.

As used herein, the term “Hx” refers to hemopexin.

As used herein, the term “ICH” refers to spontaneous intracerebral hemorrhage.

As used herein, the term “about” means plus or minus 5% of the numerical value of the number with which it is being used. Therefore, about 50% means in the range of 45%-55%.

“Administering” when used in conjunction with a therapeutic means to administer a therapeutic directly to a subject, whereby the agent positively impacts the target. “Administering” the therapeutic drug or compound may be accomplished by, for example, injection, oral administration, topical administration, or by these methods in combination with other known techniques. Such combination techniques include heating, radiation, ultrasound and the use of delivery agents. When a compound is provided in combination with one or more other active agents (e.g. other Hx attenuating or protective agents), “administration” and its variants are each understood to include concurrent and sequential provision of Hx and another compound or salt and other agents.

By “pharmaceutically acceptable” it is meant the carrier, diluent, adjuvant, or excipient must be compatible with the other ingredients of the formulation and not deleterious to the recipient thereof.

“Composition” as used herein is intended to encompass a product comprising the specified ingredients in the specified amounts, as well as any product which results, directly or indirectly, from combination of the specified ingredients in the specified amounts. Such term in relation to “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

As used herein, the term “agent,” “active agent,” “therapeutic agent,” or “therapeutic” means a compound or composition utilized to treat, combat, ameliorate, prevent or improve an unwanted condition or disease of a patient. Furthermore, the term “agent,” “active agent,” “therapeutic agent,” or “therapeutic” encompasses a combination of, for example, Hemopexin and one or more additional agent as described in the present invention.

A “therapeutically effective amount” or “effective amount” of a composition is a predetermined amount calculated to achieve the desired effect, i.e., to inhibit, block, or reverse the activation, migration, proliferation, alteration of cellular function, and to preserve the normal function of cells. The activity contemplated by the methods described herein includes both medical therapeutic and/or prophylactic treatment, as appropriate, and the compositions of the invention may be used to provide improvement in any of the conditions described. It is also contemplated that the compositions described herein may be administered to healthy subjects or individuals not exhibiting symptoms but who may be at risk of developing a particular disorder. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the chosen dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue in the amounts described in the embodiments.

The terms “treat,” “treated,” or “treating” as used herein refer to both therapeutic treatment and prophylactic or preventative measures, wherein the object is to prevent or slow down (lessen) an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes prolonging survival as compared to expected survival if not receiving treatment.

Spontaneous intracerebral hemorrhage (ICH) has an annual incidence of 21 per 100,000 persons (approximately 66,000 in USA) [4]. Although it has a high mortality rate and a high rate of disability in survivors, it has not been intensively investigated, and no specific therapies are currently available to reduce injury and improve outcome.

An immediate consequence of intracerebral hemorrhage (ICH) is the release of toxic concentrations of heme into the extravascular space. This heme is initially sequestered within erythrocytes, where it is largely prevented from participating in deleterious free radical reactions by its location within hydrophobic pockets of the hemoglobin molecule. Unfortunately, this protective structure is transient. Erythrocyte lysis and subsequent hemoglobin autoxidation are detected within a few days in ICH models. Oxidized hemoglobin (methemoglobin) has a much lower affinity for its heme moieties than reduced hemoglobin, and readily transfers them to lipids and proteins that are more vulnerable to their pro-oxidant effect. Hemin, the oxidized form of heme, is present in an evolving hematoma at concentrations that are at least 100-fold greater than the neurotoxic threshold in vitro. A substantial and growing body of experimental evidence indicates that heme/hemin release contributes to delayed peri-hematomal cell injury and resulting edema.

Erythrocyte lysis after intracerebral hemorrhage (ICH) exposes adjacent cells to toxic concentrations of hemoglobin, which rapidly oxidizes to methemoglobin and releases its heme moieties. Experimental evidence suggests that heme and its degradation products initiate oxidative and inflammatory injury cascades that may be amenable to targeted therapies. The primary defense against heme toxicity is provided by hemopexin, a glycoprotein that binds it with extraordinary affinity and mitigates its pro-oxidant and pro-inflammatory effect. A prior study demonstrated that hemopexin knockout increased striatal injury in both the collagenase and blood injection ICH models.

Our experiments have demonstrated that mice lacking Hx sustain more severe peri-hematomal injury and neurological deficits after ICH than their wild-type counterparts. However, no published studies have assessed the therapeutic potential of exogenous Hx therapy after ICH. Therefore, based on the studies described herein, Hx has a robust protective effect on perihematomal cells when administered by i.p. injection after hemorrhage and that increased plasma and/or serum concentrations of hemopexin protect the blood brain barrier from damage.

Neurotoxicity of hemoglobin. Hemoglobin (Hb) is a tetrameric molecule containing four heme groups that mediate oxygen transport. It is released from erythrocytes in the hours after CNS hemorrhage by undefined mechanisms that may be associated with complement activation [5], and may then participate in uncontrolled redox reactions. Its potent pro-oxidant effect has been observed in a number of in vitro systems [6-11]. The heme groups of Hb are sequestered in hydrophobic pockets that minimize their reactivity [12]. However, extracellular Hb is very vulnerable to oxidation to methemoglobin [13]. This process, called autoxidation, occurs in a predictable fashion after clinical ICH, and is the basis for estimating hematoma age via magnetic resonance imaging [14]. The affinity of methemoglobin for its heme moieties is relatively weak, resulting in their transfer to lipid and protein binding sites [15]. Heme and hemin, its oxidized form, are cytotoxic via at least two mechanisms. First, they are directly toxic by decomposing preformed membrane lipid peroxides to initiate free radical chain reactions [16,17]. Second, their breakdown by the heme oxygenases (HO's) releases iron, which may then catalyze formation of hydroxyl radicals via the Fenton reaction [18].

Effect of hemopexin (Hx) on heme/hemin toxicity and catabolism. Hx is an ˜60 kDa serum glycoprotein that tightly binds both heme and hemin (Kd<1 pM [19]). Synthesis is primarily hepatic. Hx has also been detected in central neurons [20,21], but this may be due to uptake rather than de novo synthesis [22]. The heme-Hx and hemin-Hx complexes bind to the LRP1 receptor, which is expressed primarily by hepatocytes and microglia/macrophages [23]. Endocytosis of the receptor-ligand complex results in degradation of most if not all hemin-Hx, followed by recycling of the receptor (but not Hx itself) to the cell membrane. Hx, like haptoglobin, is depleted in hemolytic states [24].

Direct inhibition of hemin-mediated lipid peroxidation by Hx was observed in a cell-free assay over two decades ago [16]. Exogenous Hx was subsequently reported to be protective in a rat model of cold liver storage and reperfusion [25], while Hx knockout mice sustained more renal injury after intravascular hemolysis [24], and more endothelial injury after hemin injection [26]. In the CNS, Hx knockout increased tissue injury and neurologic deficits after both ICH and ischemic stroke [21,27]. In vitro studies have indicated that this protection may be mediated by several mechanisms [19]. First, Hx reduces hemin uptake by vulnerable cells and directs it to cell populations that robustly express LRP1 and are specialized for its catabolism (i.e. macrophages/microglia, hepatocytes). Second, the hemin-Hx complex induces the antioxidant/anti-inflammatory enzyme heme oxygenase-1 [21]. Third, systemic Hx directly inhibits neutrophil migration [28]. Fourth, Hx also reduces macrophage TNF-α and IL-6 production in response to heme/hemin or lipopolysaccharide (LPS) [29,30]. The latter two mechanisms suggest that passage of Hx across an intact blood-brain barrier may not be necessary for a protective effect. Rather, Hx may act peripherally by downregulating the response of infiltrating inflammatory cells after acute CNS injury.

Testing of exogenous Hx therapy in CNS injury models has been surprisingly limited to date. Rolla et al. [31] have recently reported that human Hx administered i.p. to mice decreased the severity of experimental autoimmune encephalomyelitis (EAE). Applicants have demonstrated that mice lacking Hx sustain more severe peri-hematomal injury and neurological deficits after ICH than their wild-type counterparts. However, no published studies have assessed the therapeutic potential of exogenous Hx therapy after ICH. We have observed a robust increase in perihematomal cell viability in mice treated with human Hx beginning 2 hours after ICH induction. These results provide the first evidence that Hx is protective when administered to wild-type mice after hemorrhagic CNS injury in a translationally-relevant manner.

Evaluation of a novel and promising therapy for ICH. Accordingly, the use of Hx is the first proposal to use exogenous Hx in an ICH model. Treatment options for ICH are currently limited to surgical evacuation of the hematoma when technically feasible, reversal of anticoagulation, antihypertensive therapy, and supportive care. The inadequacy of current approaches is highlighted by grim statistics. About half of ICH patients will be dead at one month; only 10% will be living independently at this time point, and only 20% will be independent at six months [32].

Experiments

Several studies were performed in order to test the efficacy of Hx therapy in two established mouse ICH models. Indeed, the studies indicate that Hx has a robust protective effect on perihematomal cells after hemorrhage. These studies indicate that by increasing Hx levels systemically, significant protective effects are seen on perihematomal cells.

No published study has addressed the efficacy of therapy with exogenous Hx after ICH. The experiments described herein provide that Hx supports ICH therapy, because: 1) the therapeutic window is within the range of clinical feasibility, since heme/hemin release requires erythrocyte lysis over days after ICH; 2) the heme/hemin concentration in a hematoma rises to the high micromolar range (390±247 μM [33]), but plasma Hx concentration is 8-20 μM, and each Hx molecule can bind only one heme/hemin; 3) binding of heme or hemin to Hx is essentially irreversible [23], so endogenous Hx will likely be saturated; (4) recent evidence indicates that Hx also has beneficial effects on components of the inflammatory response that have been implicated in the pathogenesis of ICH, including neutrophil migration [28,34] and macrophage TNF-α and IL-6 production [29,30,35]. Given the high heme/hemin content and robust inflammatory response associated with ICH, assessment of Hx therapy is clearly warranted.

We tested highly purified human Hx from CSL Behring (King of Prussia, Pa.), as a therapy for providing beneficial effects on the blood-brain barrier and the viability of perihematomal cells after ICH. In one study, we tested Hx's effect in the mouse collagenase ICH model. Hx administered by i.p. injection beginning two hours after collagenase and repeated daily protected cells in striatal tissue adjacent to the hematoma. In an effort to facilitate Hx passage across the blood-brain barrier, we also tested the same Hx dose administered intranasally; a trend toward protection when Hx was administered intranasally was also observed, but differences in that experiment did not quite reach statistical significance. Accordingly, injection of Hx may be appropriate through several routes of administration, wherein systemically or via introduction into the brain. Furthermore, our studies support that IV administration in human patients is appropriate.

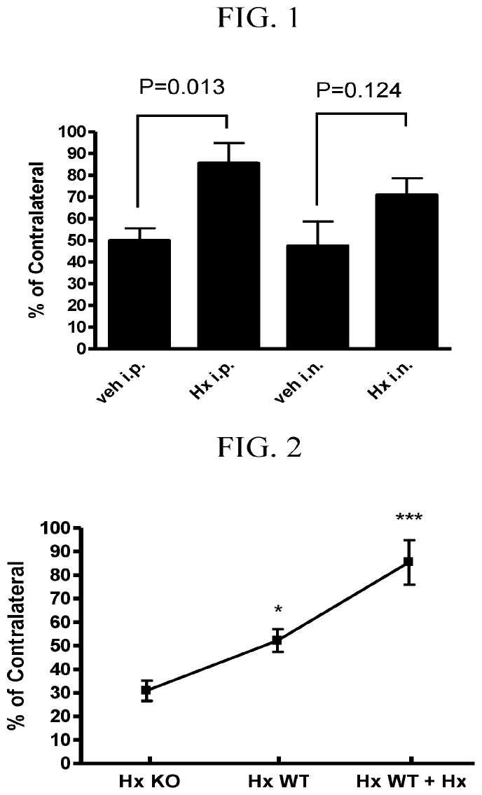

FIG. 1. Hemopexin therapy protects striatal cells after ICH. Wild-type mice received right striatal injection of collagenase followed 2 hours later by 70 mg/kg purified human hemopexin (Hx) i.p. or intranasally (i.n.) or PBS vehicle, repeated daily. Striatal cell viability was quantified by MTT assay on day 3, as previously described and validated [36], n=5/condition.

Further depicted in FIG. 1 is that hemopexin increases the viability of cells surrounding an intracerebral hematoma. Mice were treated with 70 mg/kg hemopexin i.p. or PBS vehicle 2 hours after collagenase-induced intracerebral hemorrhage, with repeated doses daily. Striatal cell viability was quantified on Day 3 by MTT assay; values are expressed as a percentage of those in the contralateral striatum. *P<0.05 v. vehicle-treated group, (n=5 per condition).

FIG. 2. Inverse relationship of perihematomal cell viability and hemopexin (Hx) level, comparing results in Hx KO mice, wild-type (WT) mice, and WT mice treated with 70 mg/kg i.p. human Hx daily beginning 2 hours after collagenase. *P<0.05, ***P<0.001 v. Hx KO mice, 5-12/condition.

Interpretation of the data. These results demonstrate for the first time that brain injury after ICH is inversely related to serum Hx level, and support that administration of Hx is useful for limiting brain injury. Accordingly, a method for limiting brain injury after ICH comprises administering to a patient an effective amount of Hx to increase serum Hx level beyond normal physiological levels to limit brain injury. One possible reason for the need for increased Hx is that Hemin binds to all of the normal Hx in the body, and thus effectively captures the Hx after ICH injury. Therefore, levels of two or three times normal physiological levels can be utilized to provide for neuro protection even in the face of ICH injury.

According to the examples and figures, therefore, administration of Hx after ICH provides for several therapeutic advantages found by no other treatment currently available. Administration of an effective amount of Hx after ICH provides for reduction in perihematomal cell injury and blood-brain barrier disruption, which will result in reduction in edema and improved outcome.

In certain aspects, timely administration of Hx to a patient after ICH provides increased benefits. Accordingly, in preferred methods, Hx is administered to a patient within about 1 to about 24 hours (and all time points in between) after ICH. In further methods, administration at up to 72 hours after ICH still provides for the perihematomal benefits to the patient.

In further methods administration of Hx is provided to a patient for at least 21 days after ICH injury, and a method comprises administration of an effective dose of Hx to increase serum Hx levels in the patient to prevent perihematomal injury to the patient, including attenuation of edema, inflammation and neurological deficits after ICH. Further methods comprise administration of Hx at least once a day, at least twice a day, and at least three times a day for between one to 21 days, with preferred treatment for between at least 3 to at least 14 days, or until the hematoma resolution in the patient.

In preferred embodiments, the serum level is raised to between two to three times physiological levels. An appropriate dose can be calculated for the individual patient based on the body mass of the particular patient with preferred doses of between about 1 to about 250 mg/kg dose. Preferred doses are given between about 10 to about 150 mg/kg, and preferred doses are about 35-100 mg/kg. In certain embodiment, it may be sufficient, however, to have a serum concentration that is approximately 1.5x, 2x, 3x, 4x, 5x or up to 10x physiologic levels. Administration of a bolus dose to quickly reach such levels is appropriate in certain embodiments and maintenance dosing over a pre-determined schedule can be utilized to maintain elevated serum concentrations.

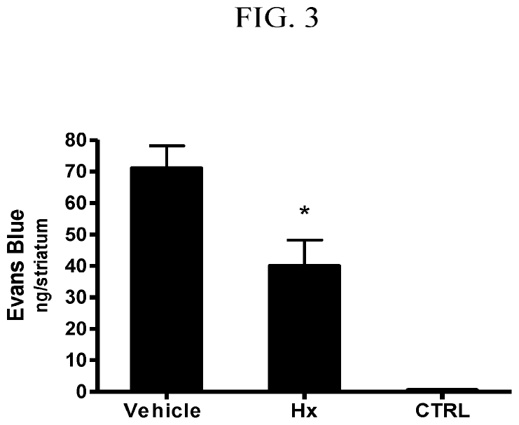

Administration and bioavailability of Hx is confirmed through several studies. Bioavailability concerns have been addressed in preliminary studies by demonstration of efficacy. The data in FIGS. 1 and 2 of our application demonstrated that hemopexin, administered by intraperitoneal (i.p) injection 2 hours after intracerebral hemorrhage (ICH), robustly increased striatal cell viability 3 days later. Recent experiments conducted in our laboratory indicate that hemopexin also protects the blood-brain barrier at this time point (FIG. 3).

Indeed, FIG. 3 depicts that hemopexin treatment after ICH attenuates blood-brain barrier injury. Mice were treated with 70 mg/kg hemopexin (Hx) i.p. daily beginning two hours after striatal collagenase injection. Blood-brain barrier integrity was assessed three days later by Evans blue assay. Control mice were subjected to surgical trauma only and thus did not have striatal collagenase injection. *P<0.05 compared with vehicle, 5-7/condition. These results indicate that systemic hemopexin therapy is protective after ICH, and provide compelling evidence that systemic administration is suitable. Indeed, after ICH injury the vehicle (PBS only) resulted in significant accumulation of Evans Blue in the striatum. In comparison, Hx administration attenuated the effects of injury to the blood brain barrier as is seen by the reduced amount of Evans Blue.

Therefore, methods for protecting the blood-brain barrier from injury suffered after ICH comprise administering a sufficient amount of hemopexin to increase serum concentrations to between two and three times physiological levels so as to attenuate blood-brain barrier injury to said patient.

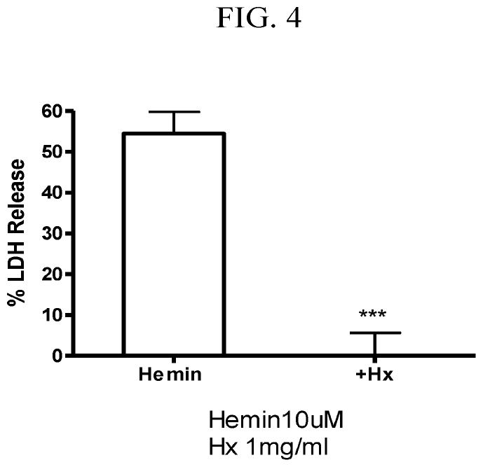

FIG. 4 depicts that hemopexin blocks the neurotoxicity of oxidized heme (hemin) in vitro. Murine cortical cultures were treated with hemin 10 μM alone or with 1 mg/ml hemopexin (Hx). Cell injury was assessed by LDH release assay. As is evident from the figure, administration of Hx dramatically reduced cell injury as compared to a control.

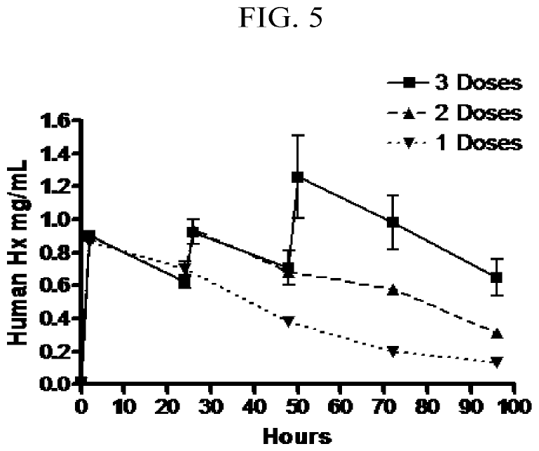

FIG. 5 depicts that Human hemopexin injection 70 mg/kg i.p. daily maintains serum concentration near target range of 0.6-1.2 mg/ml. Native mouse Hx is not measured in this assay and ranged from 0.5-1.0 mg/ml. Therefore, in preferred embodiments, the total hemopexin in the mouse was roughly 1.1-2.2 mg/ml. In administration of human Hx to a human patient, a goal is to increase serum Hx concentrations above the normal physiological levels. In preferred embodiments, the increased serum Hx concentration is roughly 1.0 to about 3.5 mg/ml. In order to maintain the serum levels at these elevated numbers, it will be necessary to provide for repeated administration of the Hx to a patient. This can be achieved through daily administration. In other embodiments, the elevated levels can also be achieved and maintained through a loading and subsequent maintenance therapeutic schedule, wherein several doses are given to achieve a predetermined serum Hx level and thereafter, subsequent administration occurs on a modified basis, such as every other day.

For example, a preferred embodiment provides for administration of Hx after the occurrence of ICH in a single bolus dose once a day for three days, followed by a maintenance dose provided on the 5^{th}, 7^{th}, 9^{th}, 11^{th}, and 13^{th} doses, and continuing until the elevated Hx levels are not necessary. Hx can be administered until hematoma resolution is achieved. In most instances this occurs within 21 days in a human patient.

Additional embodiments may use a first loading phase, consisting of doses provided to a patient about every 4, 8, 12, 16, 20, or 24 hours, until a desired serum concentration is met and maintained for at least about 4, 8, 12, 16, 20, 24, 36, or 48 hours. Preferably, this is completed with several doses administered over the course of about 1-3 days. Subsequent to the first loading phase, a maintenance phase is thereafter applied with a maintenance dose of Hx indicated for delivery to the patient on a reduced dosing schedule as compared to the first loading phase. So if the first loading phase was a dose every 12 hours, the maintenance phase would provide a dose of Hx less frequently than every 12 hours, such as every 16, 20, 24, 30, 36, 48, or 72 hours, or as determined by the pharmacokinetic profile of the patient and the half-life of the Hx in the body. In certain embodiments, a second loading phase may follow the maintenance phase, and after the second loading phase, a second maintenance phase may begin.

In further embodiments, the Hx may also be administered via a slow drip, e.g. IV, over the course of several hours or days, wherein the Hx is administered at a rate to achieve a predetermined elevated serum levels in about 12, 24, 36, 48, or 72 hours, or a time in-between those values. Subsequent to reaching the predetermined elevated serum level, a maintenance phase may commence, with a reduced Hx drip rate, or beginning Hx administration after a period of 4-72 hours, or modifying from a constant drip to a bolus injection or administration.

Several embodiments may advantageously utilize any known pumping mechanism for routine and measured administration to a patient of the drug, such as through the use of any known peristaltic pumping system for delivery of precise and small doses of fluids to a patient.

In preferred embodiments, it is important to maintain serum levels at about two to three times normal serum levels (normal serum levels are about 0.6 to about 1.2 mg/ml). It is possible to achieve these serum levels by certain mechanisms of administration to the patient. In a preferred embodiment, it is sufficient to administer the hemopexin through IV administration. Because of the nature of the hemopexin molecule, it may be necessary to co-administer or formulate the composition for entry past the blood brain barrier.

In other embodiments, direct administration to the brain is a suitable mechanism for administration. In patients suffering from ICH, ventricular catheters may be utilized to assist in maintaining intracranial pressure. Further embodiments provide for intracerebroventricular infusion. Accordingly these catheters and or other openings in the brain cavity, allow for direct administration of the hemopexin to the brain. This direct route of administration allows the drug to bypass the blood-brain barrier, and thus increased concentrations may be found with lower doses than are otherwise needed through IV administration. Indeed 35 mg/kg was tested for efficacy in certain embodiments. Accordingly, appropriate doses include 1 to 250 mg/kg for administration. Based on these concentrations, an appropriate volume can be determined to reach a predetermined serum concentration in the patient.

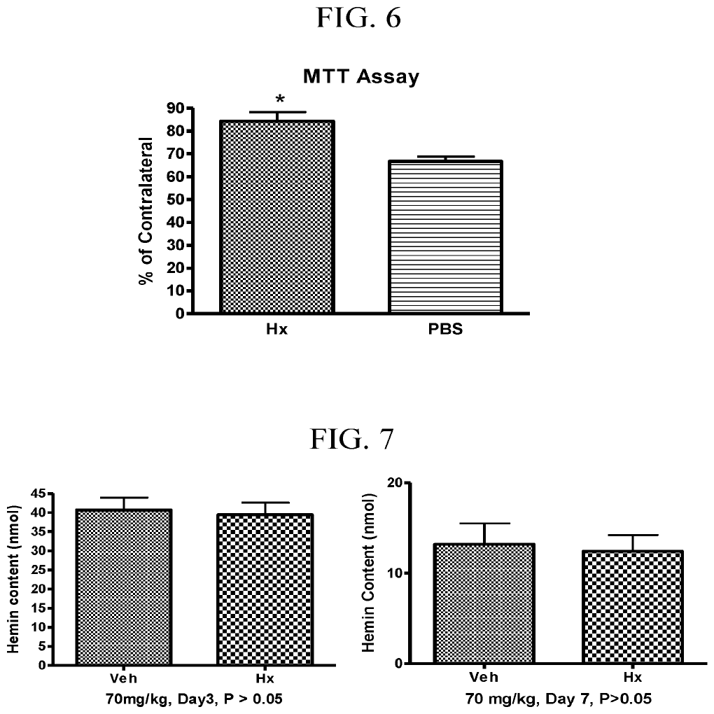

FIG. 6 depicts that lower dose Hx therapy increases cell viability in the blood injection intracerebral hemorrhage model. Mice received Hx 35 mg/kg i.p. daily beginning 2 hours after striatal blood injection, and repeated daily. Striatal cell viability was quantified at 8 days with MTT assay. *P=0.014 v. mice treated with PBS vehicle.

FIG. 7 depicts that hemopexin therapy has no effect on striatal oxidized heme (hemin) content. Mice had intracerebral hemorrhage induced by collagenase injection, then were treated daily with 70 mg/kg hemopexin or PBS vehicle. Striatal hemin was assayed at days 3 and 7. These results indicate that hemopexin is not protecting by mobilizing and removing heme from the striatum. Indeed, here remains relatively stable even with hemopexin administration. Therefore, protection of the blood brain barrier and other protective effects in the brain are achieved without significant reduction in the heme levels.

Accordingly, the present study identifies that exogenous human hemopexin protected wild-type mice in these models. Pharmacokinetic studies demonstrated that 70 mg/kg hemopexin i.p. daily maintained serum human hemopexin concentrations at 0.9-1.2 mg/ml, a range similar to human physiologic levels, and had no sustained effect on mouse hemopexin levels. Injection of collagenase into the right striatum of Swiss-Webster mice reduced perihematomal cell viability to 50±6% of contralateral, as measured by MTT assay after striatal dissociation.

Treatment with 70 mg/kg human hemopexin i.p. daily beginning two hours after collagenase increased striatal cell viability to 85±9% (P=0.013). Hemopexin at this dose also provided significant blood-brain barrier protection, with leakage of Evans blue decreasing from 71±7 to 40±8 ng/striatum. However, a lower hemopexin dose (35 mg/kg) provided no benefit for that particular protective feature.

A more variable effect was observed using C57BL/6 mice expressing the red fluorescent protein dTomato in neurons, with significant protection observed at 8 days after collagenase injection, but not at 3 days. The blood injection model produced somewhat less injury, reducing striatal cell viability to 67±2% of contralateral at three days in control mice, increasing to 84±4% with 35 mg/kg hemopexin treatment (P<0.05). These results indicate that systemic therapy with human hemopexin mitigates perihematomal cell loss after experimental ICH.

Therefore, a preferred embodiment is directed to a method for providing perihematomal protection to a patient after suffering an ICH comprising administering an effective amount of Hx to said patient to increase serum concentrations to between two and three times normal serum levels, wherein said increased serum concentrations are maintained for between 3 and 21 days.

Therefore, a method of treatment for increasing the viability of cells surrounding an intracerebral hematoma comprises administering to a patient an effective dose of hemopexin for between 1 and 21 days, wherein the hemopexin increases the viability of cells surrounding the intracerebral hematoma as compared to the viability of cells for an untreated patient.

The effective dose of administration of hemopexin can be determined by one of ordinary skill in the art. Suitable administration includes doses of between 1 and 1000 mg/kg hemopexin, which are suitably administered via IV or via direct administration to the brain cavity. Suitable dosing schedules comprise a single bolus dose in certain embodiments. In further embodiments, two, three, or more doses given in a single day are appropriate to increase serum hemopexin levels.

In preferred embodiments, hemopexin administration may continue for between one and 21 days, or longer to provide protective effects to the patient and to resolve and/or treat the hematoma.

Further embodiments are therefore envisioned wherein use of hemopexin is provided for treatment of certain conditions after ICH injury. Accordingly, it is envisioned that hemopexin can be used for reduction of perihematomal injury after ICH.

Additional uses are envisioned based on the disclosure of the invention as described herein, wherein appropriate uses of hemopexin are provided for treatment to reduce edema, reduction of inflammation, reducing neurological deficits after ICH, for reducing the breakdown of the blood-brain barrier, reduces hemin uptake by vulnerable cells and directs it to cell populations that robustly express LRP1 and are specialized for its catabolism (i.e. macrophages/microglia, hepatocytes); inducement of the antioxidant/anti-inflammatory enzyme heme oxygenase-1; inhibition of neutrophil migration; reduction of macrophage TNF-α and IL-6 production in response to heme/hemin or lipopolysaccharide (LPS).

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### LITERATURE CITED

1. Tenenbein M, Kowalski S, Sienko A, Bowden D H, Adamson I Y. Pulmonary toxic effects of continuous desferrioxamine administration in acute iron poisoning. Lancet 1992,339(8795):699-701.
2. Barnard M L, Muller-Eberhard U, Turrens J F. Protective role of hemopexin on heme-dependent lung oxidative stress. Biochem Biophys Res Commun 1993; 192(1):82-7.
3. Ghosh S, Adisa O A, Chappa P, Tan F, Jackson K A, Archer D R, Ofori-Acquah S F. Extracellular hemin crisis triggers acute chest syndrome in sickle mice. J Clin Invest 2013; 123(11):4809-20.
4. Rincon F, Mayer S A. The Epidemiology of Intracerebral Hemorrhage in the United States from 1979 to 2008. Neurocrit Care 2013;19(1):95-102.
5. Hua Y, Xi G, Keep RF, Hoff J T. Complement activation in the brain after experimental intracerebral hemorrhage. J Neurosurg 2000; 92(6):1016-1022.
6. Misra H P, Fridovich I. The generation of superoxide radical during the autoxidation of hemoglobin J. Biol. Chem. 1972; 247:6960-6962.
7. Puppo A, Halliwell B. Formation of hydroxyl radicals from hydrogen peroxide in the presence of iron: Is haemoglobin a biological Fenton reagent? Biochem. J. 1988; 249:185-190.
8. Sadrzadeh S M H, Graf E, Panter S S, Hallaway P E, Eaton J W. Hemoglobin: A biologic Fenton reagent. J. Biol. Chem. 1984; 259(23):14354-56.
9. Sadrzadeh S M H, Anderson D K, Panter S S, Hallaway P E, Eaton J W. Hemoglobin potentiates central nervous system damage. J. Clin. Invest. 1987; 79:662-664.
10. Takenaka K, Kassell N F, Foley P L, Lee K S. Oxyhemoglobin-induced cytotoxicity and arachidonic acid release in cultured bovine endothelial cells. Stroke 1993; 24(6):839-45.
11. Lamb N J, Quinlan G J, Mumby S, Evans T W, Gutteridge J M C. Haem oxygenase shows pro-oxidant activity in microsomal and cellular systems: implications for the release of low-molecular-mass iron. Biochem. J. 1999; 344:153-158.
12. Hebbel R P, Eaton J W. Pathobiology of heme interaction with the erythrocyte membrane. Sem. Hematol. 1989; 26:136-149.
13. Alyash A. Redox and radical reactions of hemoglobin solutions: toxicities and protective strategies. In: Winslow R, editor. Blood Substitutes. London: Academic Press; 2006. p 197-205.
14. Bradley W G, Jr. M R appearance of hemorrhage in the brain. Radiology 1993; 189(1):15-26.
15. Bunn H F, Jandl J H. Exchange of heme along hemoglobins and between hemoglobin and albumin. J Biol Chem 1968;243:465-475.
16. Gutteridge J M C, Smith A. Antioxidant protection by haemopexin of haem-stimulated lipid peroxidation. Biochem. J. 1988; 256:861-865.
17. Vincent S H, Grady R W, Shaklai N, Snider J M, Muller-Eberhard U. The influence of heme-binding proteins in heme-catalyzed oxidations. Arch Biochem Biophys 1988; 265(2):539-50.
18. Gutteridge J M C. Iron promoters of the Fenton reaction and lipid peroxidation can be released from haemoglobin by peroxides. FEBS Letters 1986; 201(2):291-295.
19. Tolosano E, Fagoonee S, Morello N, Vinchi F, Fiorito V. Heme scavenging and the other facets of hemopexin. Antioxid Redox Signal 2010; 12(2):305-20.
20. Tolosano E, Cutufia MA, Hirsch E, Silengo L, Altruda F. Specific expression in brain and liver driven by the hemopexin promoter in transgenic mice. Biochem Biophys Res Commun 1996; 218(3):694-703.
21. Li RC, Saleem S, Zhen G, Cao W, Zhuang H, Lee J, Smith A, Altruda F, Tolosano E, Dore S. Heme-hemopexin complex attenuates neuronal cell death and stroke damage. J Cereb Blood Flow Metab 2009; 29(5):953-64.
22. Swerts J P, Soula C, Sagot Y, Guinaudy M J, Guillemot J C, Ferrara P, Duprat A M, Cochard P. Hemopexin is synthesized in peripheral nerves but not in central nervous system and accumulates after axotomy. J Biol Chem 1992; 267(15):10596-600.
23. Hvidberg V, Maniecki MB, Jacobsen C, Hojrup P, Moller H J, Moestrup S K. Identification of the receptor scavenging hemopexin-heme complexes. Blood 2005; 106(7):2572-9.
24. Tolosano E, Hirsch E, Patrucco E, Camaschella C, Navone R, Silengo L, Altruda F. Defective recovery and severe renal damage after acute hemolysis in hemopexin-deficient mice. Blood 1999; 94(11):3906-14.
25. Brass C A, Immenschuh S, Song D X, Liem H H, Eberhard U M. Hemopexin decreases spontaneous chemiluminescence of cold preserved liver after reperfusion. Biochem Biophys Res Commun 1998; 248(3):574-7.
26. Vinchi F, Gastaldi S, Silengo L, Altruda F, Tolosano E. Hemopexin prevents endothelial damage and liver congestion in a mouse model of heme overload. Am J Pathol 2008; 173(1):289-99.
27. Chen L, Zhang X, Chen-Roetling J, Regan R F. Increased striatal injury and behavioral deficits after intracerebral hemorrhage in hemopexin knockout mice. J Neurosurg 2011; 114(4):1159-67.
28. Spiller F, Costa C, Souto F O, Vinchi F, Mestriner F L, Laure H J, Alves-Filho J C, Freitas A, Rosa J C, Ferreira S H and others. Inhibition of neutrophil migration by hemopexin leads to increased mortality due to sepsis in mice. Am J Respir Crit Care Med 2011; 183(7):922-31.
29. Liang X, Lin T, Sun G, Beasley-Topliffe L, Cavaillon J M, Warren H S. Hemopexin down-regulates LPS-induced proinflammatory cytokines from macrophages. J Leukoc Biol 2009; 86(2):229-35.
30. Lin T, Sammy F, Yang H, Thundivalappil S, Hellman J, Tracey K J, Warren H S. Identification of hemopexin as an anti-inflammatory factor that inhibits synergy of hemoglobin with HMGB1 in sterile and infectious inflammation. J Immunol 2012; 189(4).2017-22.
31. Rolla S, Ingoglia G, Bardina V, Silengo L, Altruda F, Novelli F, Tolosano E. Acute-phase protein hemopexin is a negative regulator of Th17 response and experimental autoimmune encephalomyelitis development. J Immunol 2013; 191(11):5451-9.
32. Broderick J P, Adams H P, Jr, Barsan W, Feinberg W, Feldmann E, Grotta J, Kase C, Krieger D, Mayberg M, Tilley B and others. Guidelines for the Management of Spontaneous Intracerebral Hemorrhage: A Statement for Healthcare Professionals From a Special Writing Group of the Stroke Council, American Heart Association. Stroke 1999; 30(4):905-915.
33. Letarte PB, Lieberman K, Nagatani K, Haworth R A, Odell G B, Duff T A. Hemin: levels in experimental subarachnoid hematoma and effects on dissociated vascular smooth muscle cells. J Neurosurg 1993; 79(2):252-255.
34. Zhao X, Sun G, Zhang H, Ting S M, Song S, Gonzales N, Aronowski J. Polymorphonuclear neutrophil in brain parenchyma after experimental intracerebral hemorrhage. Transl Stroke Res 2014; 5(5):554-61.
35. Hammond M D, Taylor R A, Mullen M T, Ai Y, Aguila H L, Mack M, Kasner S E, McCullough L D, Sansing L H. CCR2+ Ly6C(hi) inflammatory monocyte recruitment exacerbates acute disability following intracerebral hemorrhage. J Neurosci 2014; 34(11):3901-9.
36. Chen-Roetling J, Lu X, Regan K A, Regan R F. A rapid fluorescent method to quantify neuronal loss after experimental intracerebral hemorrhage. J Neurosci Methods 2013; 216(2): 128-36.

What is claimed is:

1. A method for administration of Hemopexin (Hx) after Intracerebral hemorrhage (ICH) comprising administering to a patient an effective amount of Hx wherein said Hx is effective in reducing perihematomal cell injury, reducing edema, reducing inflammation and reducing neurological deficits after ICH.

2. A method for treating a patient after ICH comprising administering to said patient an effective amount of hemopexin between 1 to 72 hours after suffering from said ICH.

3. The method of claim 3 wherein the Hx is administered to a patient for at least 10 days after the initial dose of hemopexin.

4. The method of claim 1 wherein the effective amount of Hx is sufficient to increase Hx serum levels to between about 2 and 3 times of the patient's normal serum Hx level.

5. The method of claim 4 comprising a first step of determining the patient's serum Hx levels so as to provide a baseline for determination of 2 and 3 times of the normal serum Hx level.

6. A method for treating a patient after ICH injury comprising administering to a patient an effective dose of Hx to increase serum Hx levels in the patient to prevent perihematomal injury to the patient, including attenuation of injury to the blood-brain barrier.

7. A method for treating a patient after ICH injury comprising administering to a patient an effective dose of Hx to increase serum Hx levels in the patient to prevent perihematomal injury to the patient, including attenuation of brain edema.

8. A method for treating a patient after ICH injury comprising administering to a patient an effective dose of Hx to increase serum Hx levels in the patient to prevent perihematomal injury to the patient, including attenuation of brain cell injury adjacent to the hematoma.

9. A method for treating a patient after ICH injury comprising administering to a patient an effective dose of Hx to increase serum Hx levels in the patient to prevent perihematomal injury to the patient, including attenuation of neurological deficits after ICH.

10. The method of any one of claims 6 through 9 comprising administration of Hx at least once a day for between one to 21 days.

11. The method of any one of claims 6 through 9 comprising administration of Hx at least twice a day for between one to 21 days.

12. The method of any one of claims 6 through 9 comprising administration of Hx at least three times a day for between one to 21 days.

13. A method of treating a hematoma in the brain comprising: determining whether an ICH injury has occurred in the brain; determining the normal serum Hx levels of the patient; determining an increased serum concentration for the patient which is between two to three times the normal serum Hx level; determining an appropriate dose of Hx wherein the increased serum concentration for the patient will be reached within 48 hours.

14. The method of claim 13 wherein the increased serum concentration is achieved through at least two administrations of Hx given over the 48 hour period.

15. The method of claim 13 wherein the increased serum concentration is achieved through three or more administrations of Hx given over the 48 hour period.

16. The method of claim 13 wherein the increased serum concentration is achieved through IV infusion of the dose.

17. A method of reducing central nervous system injury in a patient after suffering an ICH by administering an effective amount of Hx to increase serum concentration to between 1.0 and 3.5 mg/ml, wherein said Hx downregulates the response of infiltrating inflammatory cells after the acute CNS injury.

18. A method of treating a patient with Hx after suffering an ICH comprising: determining an increased serum concentration for the patient which is between two to three times the normal serum Hx level; determining an appropriate dose of Hx wherein the increased serum concentration for the patient will be reached within 48 hours; and administering the Hx to the patient to so as to reach said increased serum Hx level.

19. The method of claim 18 wherein after the increased serum Hx level is reached in said patient, a maintenance phase is entered, wherein an effective amount of Hx is administered to said patient so as to maintain said increased serum Hx level.

20. The method of claim 19, wherein said increased serum Hx level is reached by IV infusion, and wherein said maintenance phase comprises IV injections of said effective amount of Hx administered every 24 hours.

21. The method of claim 19, wherein said increased serum Hx level is reached by IV infusion, and wherein said maintenance phase comprises IV injections of said effective amount of Hx administered every 48 hours.

22. The method of claim 21 wherein said maintenance phase is at least 14 days in length.

23. A method of protecting cells around a hematoma after ICH to reduce breakdown of the blood-brain barrier, comprising:
a. administering to a patient, an effective amount of Hx sufficient to raise the serum Hx levels in the patient to between two and three times normal physiological levels;
b. measuring the serum Hx level at about 24 hours post administration; wherein the level of serum Hx is determined;
c. Treating the patient with a further dose of Hx so as to achieve serum Hx levels of between two to three times normal physiological levels.

24. The method of claim 23 wherein the Hx levels between two and three times normal physiological levels are between 1.2 and 2.5 mg/ml.

25. The method of claim 23 wherein additional doses are provided based on the dose determined in step c, and wherein said doses are maintained for up to 21 days for treatment of the hematoma.

26. A method of treatment of a patient after ICH comprising administering hemopexin to a patient so as to reduces hemin uptake by vulnerable cells and directs it to cell populations that robustly express LRP1 and are specialized for its catabolism (i.e. macrophages/microglia, hepatocytes); wherein the hemin-Hx complex induces the antioxidant/anti-inflammatory enzyme heme oxygenase-1; wherein the Hx directly inhibits neutrophil migration; and wherein the H also reduces macrophage TNF-α and IL-6 production in response to heme/hemin or lipopolysaccharide (LPS).

27. A method of treatment increasing perihematomal cell viability after ICH comprising: administering an effective amount of Hx to a patient within 24 hours of suffering from the ICH, wherein the effective amount of the Hx is sufficient to raise serum Hx concentrations to between about 1.2 and 3.5 mg/ml; wherein said increased serum Hx contentration provides for a robust increase in perihematomal cell viability after ICH induction.

28. Use of hemopexin for treatment of intracerebral hemorrhage.

29. Use of hemopexin for reducing perihematomal cell injury, reducing edema, reducing inflammation, and reducing neurological deficits after ICH.
